(19) United States
(12) Patent Application Publication
(10) Pub. No.: US 2005/0176584 A1
(43) Pub. Date: Aug. 11, 2005

(54) Mehtod for high throughput screening of plant growth regulator

(75) Inventors: Sang-Soo Kwak, Yusung-ku (KR); Haeng-Soon Lee, Taejon-si (KR); Suk-Yoon Kwon, Taejon-si (KR); Chang-Jin Kim, Taejon-si (KR); Hyang-Burm Lee, Taejon-si (KR); Sang-Han Lee, Taejon-si (KR)

Correspondence Address: Jane Massey Licata Kathleen A Tyrrell;Licata & Tyrrell, 66 East Main Street, Marlton, NJ, 08053, US

(73) Assignee: Korea Research Institute of Bioscience and Biotechnology, Taejon-si (KR)

(21) Appl. No.: 10/519,511

(22) PCT Filed: May 28, 2003
(86) PCT No.: PCT/KR03/01041

(30) Foreign Application Priority Data
Jun. 27, 2002 (KR) 10-2002-0036512

(51) Int. Cl. A01N 25/00; C12Q 1/00

(52) U.S. Cl. 504/116.1; 435/4

(57) ABSTRACT

The present invention relates to a method for high throughput screening of plant growth regulator, more particularly to the method comprising; 1) culturing phytomixotrophic cells and candidates of plant growth regulator which were added in a microwell plate, 2) treating 2,3,5-triphenyltetrazolium chloride thereto, 3) reacting thereof by adding ethanol after removing solutions from microwells, 4) transferring the reacting solution into the new microwell plate, and 5) measuring optical density with a high throughput screening reader. Since the method of the present invention can rapidly and conveniently screen many samples and can also evaluate in vivo activities of plant growth regulators, it can effectively be used as a screening method for plant growth inhibitors and activators.

### FIELD OF THE INVENTION

The present invention relates to a method for high throughput screening of plant growth regulators, more particularly to the method comprising the steps of culturing photomixotrophic cells to which candidates of plant growth regulator were added and measuring cell growth on a large scale at the same time.

### BACKGROUND

The primary evaluation on the efficacy of a drug, that is a fundamental condition for the development of a plant growth regulator (PGR) including herbicides, has been generally done by investigating the effect of a drug on plant growth by making young plants raised in a greenhouse. The said method is good for the direct investigation of plant growth inhibition but has problems of requiring much time, expense and huge amount of drugs in the early stage of efficacy evaluation.

In the meantime, to develop a medicine, following steps are generally required, that is, investigating the activity (cytotoxicity) of compounds to human cell lines in vitro, applying the compounds having the activity to test animals to detect the toxicity and efficacy of the compounds, determining candidates having excellent activity without toxicity and finally carrying out clinical tests with the candidates (Skehan et al., J Natl Cancer Inst 82: 1107, 1990). Likewise, in vitro evaluation on the activity of plant growth regulators including herbicides is primarily required for the evaluation of their efficacy. The primary in vitro investigation on the activity of herbicides used to be done by using cell-free system taken out of plants. However, the result had no consistency with that of in vivo test using plants or if any, it was far from practical use. For instance, even though a PET (photosynthetic electron transport) inhibiting compound having strong activity, confirmed by Hill reaction using thylakoid membrane, the compound did not show any herbicidal activity (Asami et al., Agric Biol Chem 51: 205-210, 1987; Sato et al., Z Naturforsch 26c: 563-568, 1991).

Plant cell culture techniques including techniques to develop plant transformants using recombinant DNA, to mass-proliferate useful plants by somatic cell culture and to mass-produce the useful materials by cell culture are the core and fundamental techniques in the field of plant biotechnology. Plant culture cells are comparatively even cell group, which the administered subjects are easily absorbed to and whose culture conditions are regulated freely and accurately. Besides, the efficacy of compounds can be measured with less expense and samples. But most plant culture cells are growing depending upon carbon source supplied from outside because differentiation and development of chloroplasts do not occurring therein. More than half of the conventional herbicides were made to target on chloroplasts including photosynthetic electron transport system. Thus, in order to screen the activity of those herbicides based on plant culture cell system, it is required to use photomixotrophic cells wherein chloroplasts are differentiated well (Dalton, Biochem Soc Trans 8: 475-477, 1980; Nishida et al., Plant Cell Physiol 21: 47-55, 1980; Sato et al., Plant Cell Rep 6: 401-404, 1987). Reports have been made that the herbicidal activity can be investigated using photomixotrophic cells by measuring cell weight, oxygen generation using oxygen electrode and ion conductivity using ion conductance meter (Sato et al., Z Naturforsch 26c: 563-568, 1991; Kwon et al., Kor J Plant Tissue Cult 26: 183-187, 1999). However, those mentioned measurements are not preferred since all the measuring takes have to be done by hand (the automation of experiment are not easy) and the experiment scale may not be reduced owing to the matters of measuring methods.

Thus, the present inventors have cultured photomixotrophic cells wherein chloroplasts are differentiated well on a microwell plate along with compounds (synthetic compound, natural compound) or natural extracts (plant extracts, culture solution of microorganism) for a while, and then added reagents generally used for the confirmation of cell viability thereto, followed by automatic measurement of the effect of those compounds on cell growth using high throughput screening reader. The present inventors have completed this invention by developing a novel screening method for plant growth regulators that investigates the activity of plants efficiently by screening large number of compounds or extracts fast and simultaneously even with small amount of samples.

### DETAILED DESCRIPTION OF PREFERRED EMBODIMENTS

The present invention provides a method for high throughput screening of plant growth regulators using photomixotrophic cells.

Further features of the present invention will appear hereinafter.

The present invention provides a method for high throughput screening of plant growth regulators in which photomixotrophic cells are cultured with candidates of plant growth regulator and then cell growth is measured simultaneously on a large scale.

In order to analyze the activity of abundant compounds to plants simultaneously and efficiently even with small amount of samples, following conditions are required:

1) Establishment of proper culture cells that can reflect the activity to plants;

2) Establishment of culture system for simultaneous screening of abundant compounds even with small amount of samples;

3) Establishment of a simple method for quantitative analysis of the activity; and

4) Establishment of an automatic evaluation system for the activity.

In the present invention, photomixotrophic cells were used as proper culture cells that can reflect the effect of plant growth regulators on plants. Photomixotrophic cells can be selected from a group consisting of Amaranthus cruentus, Asparagus officinalis, Chenopodium rubrum, Cytisus scoparius, Datura innoxia, Digitalis purpurea, Glycine max, Gossypium hirsutum, Hyoscyamus niger, Nicotiana tabacum (tobacco), Marchantia polymorpha (liverwort), Spinachia oleracea and Solanum tuberosum (Plant Tissue Culture 3: 147-155. 1986). Among them, Marchantia polymorpha or Nicotiana tabacum photomixotrophic cells are preferred.

The photomixotrophic cells of the present invention have the same chloroplast structure as higher plants have. The growth speed of those cells is very fast and the cells are uniform, so that they can be inoculated by the fixed concentration and cultured even in a microwell size plates. Plant culture cells are rather a uniform cell group which the administered substances are easily absorbed to and whose culture conditions can be regulated freely and accurately. The plant culture cells are also useful for measuring the efficacy of compounds with small amount of samples and less expense. However, most plant culture cells are heterotroph, that is, carbon source should be supplied from outside because chloroplasts are not differentiated therein. In the present invention, liverwort (Marchantia polymorpha L.) (Ohta et al., Planta 136: 229-232, 1977) and tobacco (Nicotiana tabacum cv. BY4) photomixotrophic cells (Cha et al., Korean J Bot 36: 113-120, 1993) were used since chloroplasts were differentiated well in those cells and the cells were uniform and growing fast.

The photomixotrophic cells of the present invention means photomixotrophic cultured cells (PM cells), which are the plant culture cells growing better when carbon source is supplied from outside even though they have differentiated chloroplasts. In addition to the photomixotrophic cultured cells, heterotrophic cultured cells in which chloroplasts are not differentiated or photoautotrophic cultured cells that can grow without carbon source supplied from outside since chloroplasts are differentiated therein might be the candidate for plant culture cells of the present invention. However, they are not suitable for the large scale screening of the present invention since heterotrophic cultured cells do not have differentiated chloroplasts and photoautotrophic cultured cells show very slow growth speed.

In the present invention, photomixotrophic cells were cultured in a microwell plate where candidates of a plant growth regulator were added in order to establish a culture system that makes mass screening with small amount of samples possible. Every microwell plates which have been generally used for cell culture can be used for the present invention and especially, one selected from a group consisting of 24 microwell plate, 96 microwell plate, 386 microwell plate, 960 microwell plate and 9600 microwell plate is preferably used.

In the age of nano-technology, the primary screening of compounds should give required results even with small amount of samples. In order to investigate the efficacy of plant growth regulators including herbicides, it is preferable to evaluate in vitro activity of the regulators first and then carry out in vivo tests with compounds confirmed to have the activity using plants. In this invention, plant culture cells were distributed into wells of microwell plate and cultured effectively in medium added into the wells (10-1000 μg/well). Even with a very small amount of compounds, it was possible to repeat the experiment several times. Only when the activity is detected at the level of 1 ppm in the primary screening, it proceeds with the secondary screening. Therefore, the culture scale of the present invention using microwell plate seems to be very reasonable.

For the screening of plant growth regulators, every possible substance can be used, and particularly, it is preferable to choose one from a group consisting of a synthetic compound, a pure compound including natural substances, plant extracts and extracts or fractions containing culture solution of microorganism. As treat candidates for plant growth regulator to plant culture cells, it is possible to treat different candidates at the same time, to treat a candidate with different concentrations or to treat different candidates with different concentrations simultaneously. Just one screening over a microwell plate enables to measure the effect of candidates for plant growth regulator on plant growth, for which different candidates are treated with different concentrations at the same time. The above method of the present invention ensures correct screening without experimental errors caused by individual screening or tests.

In order to establish a simple treatment method for the quantitative analysis on the activity, the present inventors added 2,3,5-triphenyltetrazolium chlorolide (referred as “TTC” hereinafter) to photomixotrophic cultured cells and measured optical density, leading to the measurement of cell growth.

It is not easy in microwell size culture (150 μl/well) to measure ion concentration of a medium or the weight of cultured cells after a given period of time from being treated with compounds since the amount of initial inoculation is under μg. In order to investigate the effect of compounds on cell growth, the present inventors used TTC compound that has been widely used for investigating cell viability and makes quantitative measurement of cell damage extent possible owing to its color reaction. When TTC compound is reacted with an enzyme of mitochondrial inner membrane, it turns into a deep red formazan by reduction (Lakon, Ber Dtsch Bot Ges 60: 299, 1942). Therefore, the undamaged cells by compounds turn into red by the reaction with TTC, but damaged cells loose color. The absorption wavelength of the converted formazan is around 490 nm. Thus, the efficacy of compounds can be evaluated simply by measuring the optical density at 490 nm. If the optical density of a group treated with compounds is lower than that of a compound untreated group, the treated compounds must inhibit plant cell growth. On the contrary, if the optical density of a group treated with compounds is higher than that of a compound untreated group, the compounds must promote plant cell growth. Therefore, the screening method of the present invention can be effectively used not only for the screening of plant growth inhibitors but also for the screening of plant growth promoters.

In order to establish an automatic system to analyze the activity of various kinds of compounds quantitatively and shortly, the present inventors used high throughput screening (HTS) reader enabling to measure the absorption wavelength of color reaction products simultaneously in a short time. As of today, in order to confirm cell viability, it was general to add solvent to cells and then homogenize, followed by centrifugation. Then, measured optical density at last. But the way was not suitable for quantitative analysis of many samples at the same time. Therefore, in the preferred embodiment of the present invention, added compounds to culture cells and stopped the culture. Then, removed medium with multi-pipette and then added a certain amount of ethanol. After a while, reaction formazan generated by TTC reaction was separated from cells, by which the efficacy of compounds could be effectively evaluated without a troublesome cell-crushing procedure (see FIG. 2).

The method for screening of plant growth regulators of the present invention comprising the following steps:

1) Culturing photomixotrophic cells in a microwell plate to which candidates for a plant growth regulator are added;

2) Treating 2,3,5-triphenyltetrazolium chlorolide thereto;

3) Reacting thereof by adding ethanol after removing solutions from the microwell plate;

4) Transferring the reacting solution of the above step 3) into a new microwell plate; and

5) Measuring optical density of the microwell plate of the above step 4) with a high throughput screening reader.

As for the step 2), the treatment time of 2,3,5-triphenyltetrazolium chlorolide is generally 3-7 hours, but 4.5-5.5 hours are preferable and 5 hour treatment is the most preferable.

As for the step 3) the ethanol is preferably 10-100% ethanol, 85-100% is more preferable and 95% ethanol is the most preferable. After adding ethanol, it is preferable to induce reaction at 70° C. for 0.1-3 hours. It is more preferable to induce reaction at 55-65° C. for 0.5-2 hours and 1 hour reaction at 60° C. is the most preferable.

In the preferred embodiments of the present invention, the present inventors investigated the effect of herbicides as candidates for a plant growth regulator on photomixotrophic cells or heterotrophic cultured cells. As a result, herbicides showed better herbicidal activity in photomixotrophic cells having well-differentiated chloroplasts than in heterotrophic cultured cells having undifferentiated chloroplasts. When synthetic compounds or natural compounds were treated to photomixotrophic cells even with low concentrations, the activity to plants was clearly detected (see Table 2 and Table 3). The screening system of the present invention is very useful for screening of plant growth regulators with compounds, plant extracts or culture solution of microorganism and for the purification of the activating substances (see Table 4-Table 7).

As explained hereinbefore, the present inventors established a system suitable for the effective evaluation on large number of compounds using less samples in a short period of time with reflecting their in vitro activity to plants well enough by using photomixotrophic cells. Again, it is possible to screen plant growth regulators with less expense in a short period of time, with the screening method of the present invention. Thus, the method can be effectively used for the development of plant growth regulators such as herbicides or growth promoters.

### BRIEF DESCRIPTION OF THE DRAWINGS

The application of the preferred embodiments of the present invention is best understood with reference to the accompanying drawings, wherein:

FIG. 1 is a set of photographs showing the callus cultures (upper panel) of Marchantia polymorpha L., Nicotiana tabacum cv. (BY4) and Oryza sativa L. cv (Taebaegbyeo) and suspension cultures (lower panel) of those in Erlenmeyer flasks;

FIG. 2 is a set of photographs showing the results of screening of plant growth regulators after subculturing Marchantia polymorpha L. cells in a 96 well plate. The upper panel represents the state before treating compounds to culture cells, the middle panel represents the state on the 7th day from treating the compounds and the lower panel represents the state on the 5th hour from treating TTC to the cells of day 7 after treating the compounds;

FIG. 3 is a set of graphs showing the amount of formazan generated in cells and media on the first and fifth hour each after treating TTC to Marchantia polymorpha L. cells of day 7 which had been treated with atrazine, a photosynthesis-inhibiting herbicide, by different concentrations (final conc.: 0, 0.1, 0.3, 1, 3, 10, 30 μM) in 12 well plates.

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Culture of Marchantia polymorpha L. and Nicotiana tabacum cv. (BY4) Photomixotrophic Cells Having Differentiated Chloroplasts

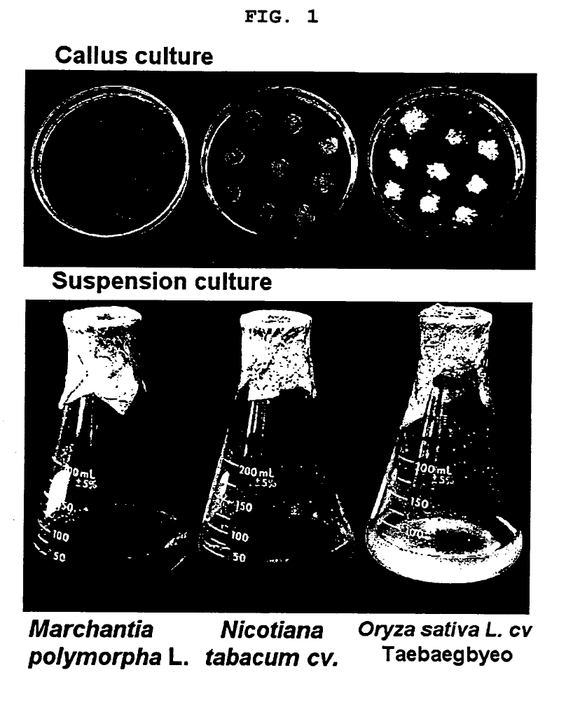

The present inventors used cells developed by Ohta et al. (Ohta et al., Planta, 136: 229-232, 1977) as Marchantia polymorpha L. photomixotrophic cells. Medium was prepared by using M51 medium (Furner et al., Plant Sci Lett, 11: 169-176, 1978) for vitamins and macronutrients, and B5 medium (Gamborg et al., Exp Cell Res, 50: 151-158, 1968) for casamino acid, glutamine and micronutrients including 2,4-D (Table 1). Suspension cultured Marchantia polymorpha L. cells were inoculated by 0.5 g into a 250 ml flask containing 50 ml of liquid medium prepared as the composition of Table 1. Then, the cells were suspension cultured at 25° C., 100 rpm under 15 μmol m⁻·s⁻¹ light condition (FIG. 1). The cells were sub-cultured at intervals of 9 days.

TABLE 1
The medium composition used for photomixotrophic cell
culture.

| Macronutrients | | | Micronutrients | | Vitamins | | Others | | |
|---|---|---|---|---|---|---|---|---|---|
| (/l) | | | (/l) | | (/l) | | (/l) | | |
| NH₄NO₃ | 400 | mg | KI | 0.75 mg | Myoino- | 100 mg | Casamino | 1 | g |
| | | | | | sitol | | no | | |
| | | | | | | | acid | | |
| KNO₃ | 2 | g | H₃BO₃ | 3 mg | Thiamine- | 10 mg | L-glutamine | 200 | mg |
| | | | | | HCl | | | | |
| CaCl₂ | 300 | mg | MnSO_{4 ·} | 10 mg | Nicotinic | 1 mg | 2,4-D | 1 | mg |
| | | | 4H₂O | | acid | | | | |
| KH₄PO₂ | 275 | mg | ZnSO_{4 ·} | 2 mg | Pyrido | 1 mg | Sucrose | 20 | g |
| | | | 7H₂O | | xine- | | | | |
| | | | | | HCl | | | | |
| MgSO₄ · | 370 | mg | CuSO_{4 ·} | 0.025 mg | FeSO_{4 ·} | 27.9 mg | | | |
| 7H₂O | | | 5H₂O | | 7H₂O | | | | |
| | | | CoCl₂ · | 0.025 mg | Na₂- | 37.3 mg | | | |
| | | | 6H₂O | | EDTA | | | | |
| | | | Na₂MoO₄ · | 0.25 mg | | | | | |
| | | | 2H₂O | | | | | | |

As Nicotiana tabacum cv. (BY4) photomixotrophic cells, the cells developed as NaCl resistance cells by Cha et al. (Cha et al., Korean J Bot, -36: 113-120, 1993) were used. For the culture of the above cells, MS minimal medium where 0.7 mg/l of 2,4-D and 0.03 mg/l of kinetin were added was used. 2 g of Nicotiana tabacum cv. (BY4) photomixotrophic cells was inoculated into a 250 ml flask containing 50 ml of liquid medium, and then suspension cultured at 25° C., 100 rpm under 15 μmol m⁻²·s⁻¹ light condition (FIG. 1). The cells were sub-cultured at intervals of 14 days.

For the preparation of non-photomixotrophic cells (Oryza sativa L. cv Taebaegbyeo), cultured cells induced from an immature embryo (Jeong et al., Korean J Plant Tissue Culture, 18: 209-214, 1991) in N6 liquid medium where 2,4-D was added by 1 mg/l. Other culture conditions were same as those for Marchantia polymorpha L. photomixotrophic cell culture.

### Example 2

### Analysis on the Effect of Photosynthesis Inhibiting Herbicides After Treating TTC During Cell Culture in Microwells

Every compounds and extracts used in this invention were dissolved in acetone, N,N-dimethylformamide (DMF), etc and then treated into each well by 1.5 μl aseptically. The final concentration of organic solvents used for dissolving each compound was adjusted to 1% (1.5 μl/150 μl).

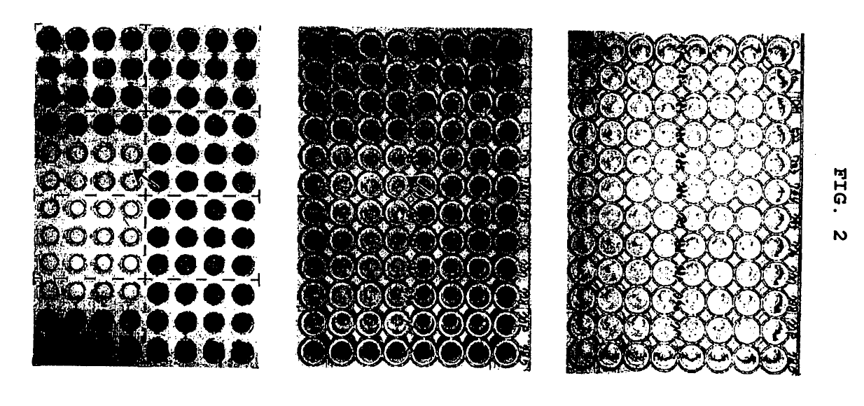

The Marchantia polymorpha L. photomixotrophic cells sub-cultured in the above Example 1 were suspended in 200 ml of liquid medium by 3 g of biomass on the 2^{nd}-3^{rd} day of subculture and then distributed into each well of a 96 microwell plate by 150 μl at the concentration of 0.2 μg biomass/150 μl/well (FIG. 2). In order to confirm whether the screening method of the present invention worked well, investigated cell viability by treating atrazine, which is a photosynthesis inhibiting herbicide working only in the presence of chloroplast, by 0, 0.1, 0.3, 1, 3, 10 and 30 μM each on the 7^{th} day of culture. And Marchantia polymorpha L. photomixotrophic cells were cultured upto be 100 ml in a flask, which were used for the evaluation of the efficacy of the compounds of the present invention.

In order to investigate cell viability on the 7^{th} day of atrazine treating culture, treated 2,3,5-triphenyl tetrazolium chlorolide (TTC) at the concentration of 12 mM. TTC compound was reacting with an enzyme of live mitochondrial inner membrane (TTC reduction), resulted in the diversion into deep red formazan (Lakon, Ber Dtsch Bot Ges, 60: 299, 1942). Therefore, undamaged cells by the treatment of the compounds turned into red color by TTC reaction, but damaged cells had no colors.

The investigation procedure of cell viability in a 96 microwell plate of the present invention is precisely explained as follows. 1) Treating 150 μl of TTC solution into each well wherein cells are being cultured. 2) Five hours after treating TTC, removing the solution in wells with a 8-channel multi-pipette. 3) Adding 150 μl of 95% ethanol to the remaining cells of each well, and then reacting thereof at 60° C. for 1 hour. 4) After reaction finished, transferring the reacting solution into a new 96 well plate. 5) Measuring the optical density of the well plate at 490 nm wave length with a high throughput screening (HTS) reader. The explained method of the present invention is called high throughput screening.

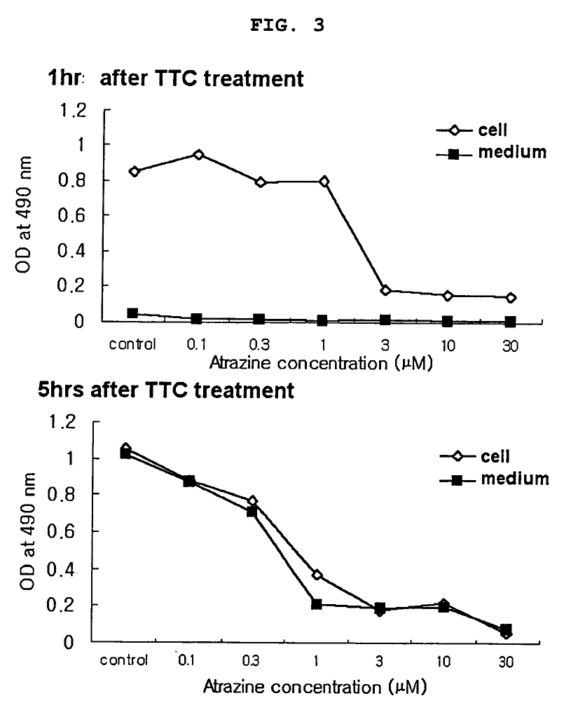

Before measuring the efficacy of the screening method using a 96 well plate, the present inventors performed TTC analysis using a 12 well plate following the same procedures as the explained high throughput screening method. Again, cultured cells in a 12 well plate where the culture solution of each well was adjusted to 1.5 ml. Treated atrazine, a photosynthesis inhibiting herbicide, by different concentrations on the 7^{th} day of culture, after which measured the amount of intracellular or extracellular formazan on the 1^{st} or 5^{th} hour from the treatment. In order to measure the amount of intracellular formazan, treated TTC first and then added 95% ethanol to the remaining cells of each well, followed by the reaction at 60° C. for an hour. Then, measured the optical density at 490 nm. In order to measure the amount of extracellular formazan, also treated TTC and then collected culture solution to measure the optical density at 490 nm. As a result, after 1 hour from the treatment of TTC, formazan in medium (extracellular formazan) was hardly detected and the content of intracellular formazan was decreased dose-dependently. After 5 hours from the treatment of TTC, both extracellular and intracellular formazan content were decreased dose-dependently. About 50% of formazan were isolated from cells, making the intracellular and extracellular formazan content almost even (FIG. 3). Thus, when TTC was treated for 5 hours, the change of intracellular formazan content was more clearly detected, which seemed to be alike with the growth inhibition curve resulted from the measurement in flask culture.

The present inventors also performed TTC analysis using a 96 well plate by the same procedure as using a 12 well plate. As a result, formazan was not separated enough from the cells 5 hours after the treatment of TTC. But, formazan content could be detected when OD of reacting solution was measured after adding 95% ethanol and further induced reaction at 60° C. for 1 hour, 5 hours after the treatment of TTC.

Every experiment using a 96 well plate included the steps of treating TTC solution for 5 hours, adding 95% ethanol thereto, reacting at 60° C. for an hour and measuring the amount of formazan generated in live cells, which offered an advantage for evaluating the efficacy of compounds easily and shortly, though with large numbers of the compounds. In the experiment using a 96 well plate, atrazine content that can inhibit cell growth upto about 50% was 0.68 μM (0.18 ppm), which was almost the same value as the cell growth inhibiting activity observed in mass-culture of cells in flasks. Thus, the inhibiting activity of compounds induced by TTC reaction reflected the cell growth inhibition pretty well.

### Example 3

### Evaluation on the Efficacy of Synthetic Compounds

The present inventors investigated the growth inhibiting activity of synthetic herbicides to Marchantia polymorpha L. photomixotrophic cells, Nicotiana tabacum cv. (BY4) photomixotrophic cells and Oryza sativa L. cv (Taebaegbyeo) heterotrophic cells using TTC analysis as performed in the above Example 2 after treating 13 kinds of conventional herbicides having different reacting mechanisms with different concentrations as stated in Table 2. Oryza sativa L. cv (Taebaegbyeo) heterotrophic cells used in this experiment were developed from premature embryo (Korean J Plant Tissue Culture, 18: 209-214, 1991).

TABLE 2
Comparison of growth inhibition activities (IC₅₀: ppm) of synthetic herbicides
to Marchantia polymorpha L. photomixotrophic cells, Nicotiana tabacum cv. (BY4)
photomixotrophic cells and Oryza sativa L. cv (Taebaegbyeo) heterotrophic cells.

| | | | Oryza | |
|---|---|---|---|---|
| | | Nicotiana | sativa L. | |
| | Marchantia | tabacum | cv | Reaction |
| | polymorpha L. | cv. (BY4) | (Taebaegbyeo) | mechanism |
| | photomixotrophic | photomixotrophic | heterotrophic | (Inhibition |
| Herbicide | cells | cells | cells | target)) |
| Atrazine | 0.21 | 0.18 | >10 | Photosynthetic |
| | | | | electron |
| | | | | transport |
| | | | | system |
| Linurone | 0.244 | >10 | 7.07 | Photosynthetic |
| | | | | electron |
| | | | | transport |
| | | | | system |
| Propanil | 0.037 | 5.83 | 8.49 | Photosynthetic |
| | | | | electron |
| | | | | transport |
| | | | | system |
| Chloromethoxynil | 0.076 | 0.193 | >10 | Protoporphyrin |
| | | | | IX |
| Oxadiazone | 0.03 | 0.39 | 6.88 | Protoporphyrin |
| | | | | IX |
| Diflufenican | 0.031 | 4.67 | >10 | Carotenoid |
| | | | | synthesis |
| Dithiopyr | 0.048 | 0.141 | 0.613 | Lipid |
| | | | | synthesis |
| Phenoxapro- | 0.70 | 4.58 | 6.47 | ACCase |
| P-ethyl | | | | synthesis |
| Amazapyr | 0.35 | 0.018 | 0.269 | Acetolactate |
| | | | | synthase |
| | | | | Synthesis |
| LGC-42153 | 0.0011 | 0.0022 | 1.793 | Acetolactate |
| | | | | synthase |
| | | | | Synthesis |
| Prazolsulfuron- | 0.0012 | 0.001 | 0.066 | Acetolactate |
| ethyl | | | | synthase |
| | | | | Synthesis |
| Pyribenzoxim | 0.0092 | 0.0026 | 7.83 | Acetolactate |
| | | | | synthase |
| | | | | Synthesis |
| Naproanilide | 0.054 | 0.0019 | 6.69 | Acetolactate |
| | | | | synthase |
| | | | | Synthesis |

In the above table, ACCase is 1-aminocyclopropane-1-carboxylic acid synthase.

As a result, LGC-42153, amazapyr, prazolsulfuronethyl, pyribenzoxim and naproanilide which are the herbicides having acetolactate synthase (ALS) inhibiting activity showed high growth inhibiting activity even with small amount in photomixotrophic cells. Especially herbicides targeting chloroplasts were proved to have strong growth inhibiting activity to photomixotrophic cells and following herbicides ought to be included in that category; 1) atrazine, linurone and propanil which inhibit photosynthetic II (PSII), 2) chloromethoxynil and oxadiazone which inhibit synthesis of protoporphyrin IX (protox), an intermediate of chlorophyll, 3) diflufenican which inhibits carotenoid synthesis. Among those herbicides, atrazine, chloromethoxynil and diflufenican showed low growth inhibiting activity or almost none even with 10 ppm to non-photomixotrophic (heterotrophic) cells (Table 2). The effects of herbicides on the market on culture cells were various. And plant growth inhibiting activities of those were better detected in photomixotrophic cells having well-differentiated chloroplasts, comparing with heterotrophic cells whose chloroplasts were not differentiated. Therefore, the method to measure formazan generated by TTC reaction with HTS reader, after culturing photomixotrophic cells in a 96 well plate, was confirmed to be very effective to measure weeding activity of the compounds.

### Example 4

### Evaluation on the Efficacy of Natural Compounds

In order to investigate the growth inhibiting activity to Marchantia polymorpha L. photomixotrophic cells of natural compounds isolated from various plants (Table 3), the present inventors performed the efficacy evaluation test used in the above Example 2, and then compared the results with the growth inhibiting activity to Lemna paucicostata, an aquatic plant (Korea Research Institute of Chemical Technology). Adjusted the final concentrations of the compounds treated to Marchantia polymorpha L. photomixotrophic cells to 1 ppm and adjusted the final concentrations of the compounds treated to Lemna paucicostata to 31 ppm.

Measured the growth inhibiting activity of the compounds to Lemna paucicostata as follows; Cultured Lemna paucicostata in a 24 well plate containing Hutner's nutrient medium, which was treated with 16 different natural compounds stated in Table 3. Investigated the growth inhibiting activity 5 days after treating. For the evaluation of the inhibiting activity, classified the grades into 6 from 0 to 5 based on the naked eye distinction. Grade 0 was defined as having under 10% inhibiting activity, so was grade 1 as having 11-30% activity, grade 3 as having 51-70% activity, grade 4 as having 71-90% activity, and grade 5 as having 91-100%. inhibiting activity. Investigated the growth inhibiting activities of the compounds to Marchantia polymorpha L. photomixotrophic cells using the same method as in Example 2. Calculated the inhibiting activity (% inhibiting activity) by 1−(OD of compound-treated group/OD of control)×100. + value means the cell growth inhibiting activity and − value means the cell growth promoting activity.

As a result, the 16 different compounds used in this invention were confirmed to have growth inhibiting activity to Marchantia polymorpha L. photomixotrophic cells though it varied, among which coumarine showed the strongest growth inhibiting activity (33%). The growth inhibiting activity to Marchantia polymorpha L. photomixotrophic cells was in proportion to the activity to Lemna paucicostata, one of aquatic plant. Interestingly, ferulic acid, dicumarol and 3-coumaranone were proved to have the growth promoting activity to Marchantia polymorpha L. photomixotrophic cells (Table 3). Marchantia polymorpha L. photomixotrophic cells were easily affected by natural compounds even with low concentration, meaning that small amount of treated natural compounds could inhibit the cell growth, making them a useful candidate for the method for screening of plant growth regulators of the present invention.

TABLE 3
Growth inhibition activities of 16 compounds isolated from
plants to Marchantia polymorpha L. photomixotrophic cells and
Lemna paucicostata.

| | Marchantia | |
|---|---|---|
| | polymorpha L. | |
| | photomixotrophic | Lemna |
| | cells | paucicostata |
| Compounds | (% activity) | (% activity) |
| Benzoic acid | 9 | 10 |
| Caffeic acid | 21 | 30 |
| Coumarin | 33 | 80 |
| Dicumarol | −31 | 30 |
| o-Coumaric acid | 32 | 30 |
| p-Coumaric acid | 17 | 30 |
| 3-Coumaranone | −18 | 0 |
| Ferulic acid | −35 | 20 |
| Gallic acid | 8 | 0 |
| Gentistic acid | 9 | 0 |
| Hydroquinone | 15 | 0 |
| Protocatechuic | 22 | 70 |
| acid ethyl ester | | |
| Scopoletin | 16 | 0 |
| Syringic acid | 16 | 0 |
| Umbelliferone | 6 | 40 |
| Vanillic acid | 10 | 10 |

### Example 5

### Evaluation on the Efficacy of Plant Extracts (Fractions)

The present inventors investigated the effect of methanol extracts (final conc.: 10 ppm) extracted from 49 kinds of plants including fruits of Viburnum dilatatum provided by Korea Plant Extract Bank of Plant Diversity Research Center on Marchantia polymorpha L. photomixotrophic cells with the same method used in the above Example 2.

As a result, the effects of those plant extracts on plant cell growth were varied. The extracts of Daphniphylium macropodum small branch, Ribes fasclculatum var. Chinese fruit, Valeriana officinalis var. latifolia leaf and trunk/root and Trichosanthes kinilowii var. japonica seed were confirmed to have more than 50% growth inhibiting activity to Marchantia polymorpha L. photomixotrophic cells (Table 4 and Table 5).

TABLE 4
The effect of 49 kinds of plant extracts on the growth
of Marchantia polymorpha L. photomixotrophic cells.

| | | | Inhibition |
|---|---|---|---|
| Plant name | | | activity |
| (Scientific name) | Region | OD₄₉₀ | (%) |
| Control | | 0.808 | 0 |
| Viburnum dilatatum | Fruit | 0.536 | 34 |
| Ilex integra | Leaf | 0.735 | 9 |
| Ilex integra | Trunk- | 0.680 | 16 |
| | heartwood | | |
| Ilex integra | Trunk-bark | 0.636 | 21 |
| Quercus gilva | Leaf | 0.756 | 6 |
| Quercus gilva | Trunk- | 0.735 | 9 |
| | heartwood | | |
| Quercus gilva | Trunk-bark | 0.841 | 0 |
| Cayratia japonica | Leaf | 0.802 | 1 |
| Cayratia japonica | Fruit | 0.623 | 23 |
| Celtis choseniana | Leaf | 0.560 | 31 |
| Celtis choseniana | Trunk | 0.570 | 29 |
| Celtis choseniana | Fruit | 0.668 | 17 |
| Staphylea bumalda | Fruit | 0.695 | 14 |
| Staphylea bumalda | Pericarp | 0.594 | 26 |
| Wasabia koreana | Root | 0.671 | 17 |
| Ligustrum japonicum | Leaf | 0.758 | 6 |
| Ligustrum japonicum | Small | 0.619 | 23 |
| | branch | | |
| Castanopsis | Leaf | 0.564 | 30 |
| cuspidata var. | | | |
| sieboldii | | | |
| Castanopsis | Trunk- | 0.530 | 34 |
| cuspidata var. | heartwood | | |
| sieboldii | | | |
| Castanopsis | Trunk-bark | 0.708 | 12 |
| cuspidata var. | | | |
| sieboldii | | | |
| Dephniphyllum | Leaf | 0.602 | 25 |
| macropodum | | | |
| Dephniphyllum | Trunk | 0.619 | 23 |
| macropodum | | | |
| Dephniphyllum | Fruit | 0.692 | 14 |
| macropodum | | | |
| Dephniphyllum | Leaf | 0.445 | 45 |
| macropodum | | | |
| Dephniphyllum | Small | 0.380 | 53 |
| macropodum | branch | | |

TABLE 5
The effect of 49 kinds of plant extracts on the growth
of Marchantia polymorpha L. photomixotrophic cells.

| | | | Inhibition |
|---|---|---|---|
| Plant name | | | activity |
| (Scientific name) | Region | OD₄₉₀ | (%) |
| Ribes fasciculatum | Fruit | 0.356 | 56 |
| var. chinense | | | |
| Ribes fasciculatum | Trunk | 0.428 | 47 |
| var. chinense | | | |
| Litsea japonica | Leaf | 0.642 | 21 |
| Litsea japonica | Trunk- | 0.499 | 38 |
| | heartwood | | |
| Litsea japonica | Trunk-bark | 0.573 | 29 |
| Catalpa | Fruit | 0.409 | 49 |
| bignonioides | | | |
| Valeriana | Leaf | 0.371 | 54 |
| officinallis var. | | | |
| latifolia | | | |
| Valeriana | Trunk, | 0.364 | 55 |
| officinallis var. | root | | |
| latifolia | | | |
| Trichosanthes | Seed | 0.379 | 53 |
| kinilowii var. | | | |
| jponica | | | |
| Trichosanthes | Sarcocarp | 0.409 | 49 |
| kinilowii var. | | | |
| jponica | | | |
| Cinnamomum camphora | Trunk- | 0.484 | 40 |
| | heartwood | | |
| Cinnamomum camphora | Trunk-bark | 0.620 | 23 |
| Clerodendrum | Leaf | 0.481 | 40 |
| trichotomum | | | |
| Clerodendrum | Trunk | 0.635 | 21 |
| trichotomum | | | |
| Carpesium | Leaf | 0.644 | 20 |
| abrotanoides | | | |
| Carpesium | Trunk | 0.749 | 7 |
| abrotanoides | | | |
| Carpesium | Root | 0.765 | 5 |
| abrotanoides | | | |
| Elaeocarpus | Leaf | 0.657 | 19 |
| sylvestris var. | | | |
| ellipticus | | | |
| Cocculus trilobus | Leaf | 0.639 | 21 |
| Cocculus trilobus | Fruit | 0.628 | 22 |
| Viburmum erosum | Fruit | 0.668 | 17 |
| Aralia | Leaf | 0.696 | 14 |
| continentalis | | | |
| Aralia | Trunk | 0.672 | 17 |
| continentalis | | | |
| Aralia | Fruit | 0.626 | 23 |
| continentalis | | | |

As seen hereinbefore, the photomixotrophic cells were available for the investigation of the activities of synthetic or natural compounds and of plant extracts as well. Therefore, it is highly expected that the HTS system of the present invention based on the use of photomixotrophic cells, can be effectively used for screening plant growth regulators and activators.

### Example 6

### Evaluation on the Efficacy of Microorganism Culture Solution

The present inventors investigated the growth inhibiting activity of culture solution of Actinomycetes spp. isolated from soil with the method used in Example 2. Then, the results were compared with results of pot test using Lemna paucicostata and other weeds. Actinomycetes spp. used in this invention was identified after thorough examination of microorganisms separated from soils everywhere in Korea and was named as stated in Table 6 and Table 7. The autoclaved culture solution used here was prepared by autoclaving the culture solution at 121° C. for 5 hours and treated with 1 ppm. In order to prepare ethyl acetate (EtOAc) extracts from culture solution, mixed 1 ml of culture solution and 1 ml of ethyl acetate, and then centrifuged. Removed the lower layer (water layer), concentrated the obtained ethyl acetate layer and treated the concentrated ethyl acetate with 10 ppm. In order to investigate the growth inhibiting activity of the microorganism culture solution to Lemna paucicostata, treated the unsterilized microorganism culture solution thereto by 31 ppm. The test procedure was same as performed in Example 4. And for the pot test in a green house, sowed Abutilon avicennae Velvetleaf, Aeschynomene indica, Agropyron smithii, Calystegia japonica, Digitaria sanguinalis, Echinochloa crus-galli, Monochoria vaginalis, Oryzae sativa, Panicum dichotomiflorum, Sagittaria pygmaea, Scirpus juncoides, Solanum nigrum, Sorghum bicolor, Trifolium repens and Xanthium strumarium in test pots (350 cm²), and cultivated according to the conventional method. Treated the unsterilized microorganism culture solution (0.1% tween 20 solution, final concentration was adjusted to 40 kg/ha) to the leaves of all the above mentioned plants using a hand sprayer. Cultivated them in a green house for 2 weeks, after which performed pot test. The weeding activity was classified into 6 grades by the pot test based on the naked eye investigation on the morphological and physiological aspects. That is, grade 0 suggests that the inhibiting activity is under 10%, likewise, grade 1 means 20-30% inhibiting activity, grade 2 suggests 40-50% inhibiting activity, grade 3 suggests 60-70% inhibiting activity, grade 4 suggests 80-90% inhibiting activity and grade 5 suggests 100% complete inhibiting activity. The classification was made by the weeding activity to at least a kind of plant. The inhibiting activity value was obtained by averaging the inhibiting activities to all the plants.

As a result, when Marchantia polymorpha L. photomixotrophic cells were treated with 1 ppm of autoclaved microorganism culture solutions, two strains (M531 and M774) showed 91% inhibiting activity and other 14 strains showed over 30% inhibiting activity. When Marchantia polymorpha L. photomixotrophic cells were treated with 10 ppm of ethyl acetate extracts, 9 strains (G715, G747, G774, G793, M690, M715, M755, M774) showed over 90% inhibiting activity and other 31 strains did more than 30% inhibiting activity. But interestingly, the autoclaved culture solutions of 32 strains including M715, M912, G745, M755, M281, etc. rather promoted cell growth, making those strains as useful candidates for plant growth stimulators. The growth inhibiting activity to Marchantia polymorpha L. photomixotrophic cells was closely related with the results of pot test with Lemna paucicostata and plants. That is, a compound confirmed to have inhibiting activity by pot test showed similar activity when used in Marchantia polymorpha L. photomixotrophic cells. Culture solutions of 42 strains did not have inhibiting activity to any of Marchantia polymorpha L. photomixotrophic cells, Lemna paucicostata and plants (Table 6 and Table 7). Therefore, the HTS system using photomixotrophic cells developed by the present inventors has been proved to be a very useful method for screening plant growth regulators in pure compounds, plant extracts, microorganism culture solutions and culture extracts.

TABLE 6
The activity of soil Actinomycetes spp. culture solution to Marchantia
polymorpha L. photomixotrophic cells, Lemna paucicostata and pot test.

| | Marchantia | | | |
|---|---|---|---|---|
| | polymorpha | | | |
| | L. photomixotrophic | | | |
| | cells | | | |
| | Autoclaved | Ethyl | | |
| | culture | acetate | Lemna | |
| Strain | solution | extracts | paucicostata | Pot test |
| G247 | −28 | 18 | 0 | 0 |
| G280 | 45 | 85 | 0 | 0 |
| G285 | 32 | 63 | 5 | 5 |
| G297 | −47 | 2 | 0 | 0 |
| G325 | −5 | −12 | 3 | 0 |
| G326 | −31 | −9 | 0 | 0 |
| G360 | −13 | 33 | 0 | 0 |
| G370 | 0 | 0 | 1 | 0 |
| G373 | 13 | 35 | 3 | 0 |
| G408 | −2 | 24 | 0 | 3 |
| G410 | 23 | 51 | 2 | 0 |
| G411 | −17 | 57 | 0 | 0 |
| G450 | −42 | 40 | 0 | 4 |
| G451 | −35 | 2 | 0 | 4 |
| G542 | 24 | −30 | 0 | 0 |
| G615 | −11 | 46 | 0 | 5 |
| G652 | 32 | 55 | 0 | 0 |
| G669 | 40 | −17 | 5 | 0 |
| G715 | 45 | 96 | 5 | 3 |
| G719 | −181 | −4 | 3 | 0 |
| G745 | −148 | −14 | 4 | 0 |
| G747 | 47 | 99 | 0 | 4 |
| G765 | −7 | −8 | 4 | 0 |
| G774 | −49 | 92 | 4 | 0 |
| G793 | −13 | 90 | 0 | 0 |
| G860 | −35 | 2 | 4 | 0 |
| G883 | 49 | −3 | 4 | 0 |
| G1160 | 25 | 47 | 4 | 0 |
| G1175 | 21 | 30 | 4 | 0 |

TABLE 7
The activity of soil Actinomycetes spp. culture solution to Marchantia
polymorpha L. photomixotrophic cells, Lemna paucicostata and pot test.

| | Marchantia | | | |
|---|---|---|---|---|
| | polymorpha | | | |
| | L. photomixotrophic | | | |
| | cells | | | |
| | Autoclaved | Ethyl | | |
| | culture | acetate | Lemna | |
| Strain | solution | extracts | paucicostata | Pot test |
| M252 | −50 | 77 | 4 | 0 |
| M253 | 7 | −34 | 4 | 0 |
| M261 | 32 | 46 | 0 | 0 |
| M281 | −133 | 93 | 0 | 0 |
| M360 | −68 | 53 | 0 | 0 |
| M366 | −22 | 30 | 0 | 0 |
| M407 | −45 | 9 | 0 | 0 |
| M413 | 21 | 15 | 3 | 0 |
| M443 | −101 | −2 | 1 | 0 |
| M447 | −31 | 14 | 3 | 5 |
| M453 | −39 | 36 | 2 | 0 |
| M531 | 91 | 76 | 0 | 0 |
| M537 | 39 | 58 | 0 | 5 |
| M533 | 9 | 2 | 0 | 0 |
| M621 | 22 | 14 | 0 | 0 |
| M635 | 38 | −13 | 4 | 0 |
| M656 | −11 | 49 | 0 | 5 |
| M690 | −9 | 94 | 4 | 5 |
| M702 | 4 | −12 | 0 | 0 |
| M705 | 50 | 52 | 4 | 4 |
| M715 | −184 | 97 | 5 | 5 |
| M745 | −129 | −84 | 4 | 0 |
| M752 | 8 | 14 | 0 | 3 |
| M755 | −146 | 96 | 0 | 5 |
| M774 | 91 | 99 | 4 | 5 |
| M787 | −65 | 32 | 0 | 0 |
| M912 | −148 | −12 | 4 | 0 |
| M938 | −81 | 92 | 5 | 0 |
| M1370 | 61 | 42 | 0 | 5 |

In the above Table 6 and Table 7,

% activity of Marchantia polymorpha L.=[1−(OD of compound treated group/OD of control)]×100,

+ value means that the cell growth inhibiting activity is detected, and − value means that the cell growth promoting activity is detected.

### INDUSTRIAL APPLICABILITY

As explained hereinbefore, the screening method of the present invention comprising the steps of culturing photomixotrophic cells having well differentiated chloroplasts in a microwell plate to which synthetic compounds, natural compounds or natural extracts are added, adding reagents thereto and measuring the cell growth using high throughput screening reader, can offer a great advantage for the evaluation of the efficacy of the compounds by analyzing various compounds shortly and easily even with small amount of the compounds. Therefore, the high throughput screening method of the present invention can be effectively used for the screening and the development of plant growth regulators with less expense in a short period of time.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for high throughput screening of plant growth regulators comprising the steps of culturing photomixotrophic cells to which candidates for plant growth regulators were added and measuring cell growth on a large scale at the same time.

2. The method as set forth in claim 1, wherein the photomixotrophic cells are Marchantia polymorpha L. photomixotrophic cells or Nicotiana tabacum cv. BY4 photomixotrophic cells.

3. The method as set forth in claim 1, wherein the candidates for plant growth regulators are selected from a group consisting of synthetic compounds, natural compounds, plant extracts and fractions or extracts containing microorganism culture solutions.

4. The method as set forth in claim 1, wherein the culture is carried out in microwell plates.

5. The method as set forth in claim 1, wherein the cell growth measurement is carried out by measuring optical density after treating 2,3,5-triphenyltetrazolium chlorolide to culture cells.

6. The method as set forth in claim 1, wherein the method comprises the following steps:
1) Culturing photomixotrophic cells in a microwell plate to which candidates for plant growth regulators are added;
2) Treating 2,3,5-triphenyltetrazolium chlorolide thereto;
3) Reacting thereof by adding ethanol after removing solutions from the microwell plate;
4) Transferring the reacting solution of the above step 3) into a new microwell plate; and
5) Measuring optical density of the microwell plate of the above step 4) with a high throughput screening reader.

7. The method as set forth in claim 6, wherein the step 3 is carried out by treating 2,3,5-triphenyltetrazolium chlorolide for 4.5-5.5 hours, removing solutions from microwells, adding 95% ethanol thereto, and then reacting thereof at 60° C. for 1 hour.
